(19) United States
(12) Patent Application Publication
(10) Pub. No.: US 2010/0159012 A1
(43) Pub. Date: Jun. 24, 2010

(54) Conjugates of Therapeutically Active Compounds

(75) Inventors: Abraham J. Domb, Efrat (IL); Itzhack Polacheck, Jerusalem (IL); Marina Sokolsky, Rishon Le Zion (IL); Jacob Golenser, Mevaseret-Zion (IL)

Correspondence Address: THE NATH LAW GROUP, 112 South West Street, Alexandria, VA, 22314, US

(73) Assignees: HADASIT MEDICAL RESEARCH SERVICES & DEVELOPMENT LIMITED, Jerusalem (IL); YISSUM RESEARCH DEVELOPMENT COMPANY OF THE HEBREW UNIVERSITY OF JERUSALEM, Jerusalem (IL)

(21) Appl. No.: 11/992,298

(22) PCT Filed: Sep. 26, 2006
(86) PCT No.: PCT/IL2006/001118

§ 371 Date: Dec. 8, 2009

Related U.S. Application Data
(60) Provisional application No. 60/719,175, filed on Sep. 22, 2005.

(51) Int. Cl. A61K 9/14; C07H 3/00; C07H 3/02; C08B 37/02; C07H 1/00; A61K 31/715

(52) U.S. Cl. 424/488; 536/123.1; 536/1.11; 536/56; 536/114; 530/300; 530/350; 536/112; 536/125; 514/54

(57) ABSTRACT

The present invention discloses modified polymer conjugates of a polymer and a drug having reduced toxicity relative to the unmodified parent compound while retaining substantially the same degree of therapeutic activity as of the unmodified parent compound.

### FIELD OF THE INVENTION

This invention relates to conjugates of therapeutically active compounds with polysaccharides.

### BACKGROUND OF THE INVENTION

Bioactive agents that exhibit limited solubility and stability or possess high toxicity may be chemically modified by conjugation to hydrophilic polymers such as polysaccharides as means to overcome these limitations and reduce their toxicity. Other methods involve formulating the bioactive drug in less toxic forms. One such example is the polyene antibiotic Amphotericin B (AmB), which is presently available in a less toxic micellar form of sodium deoxycholate-AmB (Fungizone®), as a liposomal formulation (AmBisome®), as a colloidal dispersion (Amphotec®) and as a lipid complex (Abelcet®). While the micellar form exhibits overall reduced toxicity, certain toxicity to the kidneys, central nervous system and liver alongside therapeutic limitations such as low tolerated dose still remains.

Development of water-soluble polymer-drug conjugates is pursued as a mean for achieving a targeted drug delivery and lower drug toxicity due to different organ distribution and lower accumulation in the liver and kidneys. U.S. Pat. Nos. 5,567,685 and 6,011,008 to the inventors of the present application disclose water-soluble polysaccharide conjugates of oxidation-sensitive bioactive substances, each containing a certain degree of free aldehyde groups and active moieties capable of imparting the desired therapeutic action. The inventors have recently realized that while the conjugates are therapeutically effective, a certain degree of toxicity remains.

It has been known that small molecules that carry aldehyde groups tend to be toxic. This toxicity is usually attributed to the tendency of aldehyde groups to react with amines, and thus interfere with the structure of proteins and nucleic acids. Nonetheless, there are aldehyde-containing molecules, which are known in the art to be biocompatible.

The reduction of aldehyde-stemming toxicity may be achieved by converting the aldehyde moieties into substantially less toxic groups. However, in molecules where such chemical modifications may also affect the bioactive moieties e.g. AmB, a reduction in the therapeutic action is also observed.

The balance between the reduction in toxicity imparted by the aldehyde moieties and the retention of the therapeutic action is clearly the impediment for further development of such compounds.

### SUMMARY OF THE INVENTION

It is therefore an objective of the present invention to provide modified polymer conjugates of a polymer and a therapeutically active compound, herein referred to as the drug, said conjugate having reduced toxicity relative to the unmodified parent compound while retaining substantially the same degree of therapeutic activity as of the unmodified parent compound.

The conjugates of the present invention are typically prepared from suitable precursors such as the aldehyde-containing conjugates disclosed in U.S. Pat. Nos. 5,567,685 and 6,011,008 to the inventors of the present invention. As will be further disclosed, these precursor conjugates having a plurality of aldehyde groups, herein referred to as the “parent conjugates” or “unmodified conjugates”, are chemically modified under selective conditions to chemically transform each of said aldehyde groups into a group different from —CH₂OH. The group being different from —CH₂OH may be selected in a non-limiting manner from ethers, esters, amines, imines, amides, acetals or hemiacetals as will be disclosed herein next.

Thus, the reduced aldehyde-free conjugates of U.S. Pat. Nos. 5,567,685 and 6,011,008 are hereby excluded from the scope of the present invention.

The conjugates of the invention may be characterized as follows:
1. the therapeutically active drug is conjugated to the polymer backbone via a C_{polymer}—O_{drug} or C_{polymer}—N_{drug} bond;
2. the conjugates are substantially free of aldehyde groups;
3. the conjugates have reduced toxicity in comparison with the unmodified conjugates;
4. the conjugates retain the biological and/or therapeutic activity associated with the unmodified conjugates;
5. the conjugates retain the structure of the drug conjugated to the polymer; and
6. the conjugates retain most of the physical and chemical characteristics which allow the use thereof in a fashion similar to the use of the unmodified conjugates.

The term “conjugate” as used herein, refers to a compound comprising a polymer, preferably a polysaccharide, and a drug chemically bonded (i.e. conjugated) thereto. The chemical bonding is preferably covalent bonding, most preferably via an N or O atom of the drug molecule and a C atom of the polymer, said N or O atom being an inherent part of the structure of said drug or appended thereto following chemical modifications.

In the context of the present invention the term “polymer” refers to a compound having at least one repeating monomer, and a molecular weight of at least 1,000 Dalton, preferably at least 10,000 Dalton, and more preferably in the range of 5,000 to 75,000 Daltons. The polymers employed may be linear or branched. In case the polymer is constructed of at least two repeating monomers, the polymer may be ordered, e.g. having an alternating sequence of each of the at least two monomers, or may be constructed in a random unordered fashion. Thus, the term “polymer” also includes homopolymers, copolymers, terpolymers, and higher polymers.

As will be shown next, the conjugate of the invention is prepared by partially oxidizing a polymer to afford a partially oxidized polymer having a plurality of oxidized monomers. The oxidized monomers of the polymer are then modified in accordance with the present invention to afford a polymer having three different monomers: (i) a non-oxidized monomer which retains its original structure, (ii) a drug-bearing aldehyde-free monomer, and (iii) a drug-free and aldehyde-free monomer.

In a preferred embodiment, said polymer is a polysaccharide having repeating monosaccharide units which may be the same (such as in the case of dextran) or may be different (such as in the case of arabinogalactan), said polysaccharide may be natural or synthetic and may be either branched or linear. The polysaccharide may also be synthetically modified natural polysaccharide. Preferably, said polysaccharide is selected from water-soluble or water-dispersible polysaccharides.

Non-limiting examples of polysaccharides are starch (composed of a combination of the polysaccarides amylose and amylopectin), glycogen (a branched polysaccharide composed of repeating glucose monomers), cellulose (composed of repeating glucose units bonded together via n-linkages), dextran (a linear polysaccharide composed of repeating glucose units), pullulan (composed of repeating maltotriose monomers), chitosan (composed of distributed β-(1-4)-linked D-glucosamine and N-acetyl-D-glucosamine units), arabinogalactan (AG, a branched natural polysaccharide composed of galactose and arabinose units linked together in a ratio of 6 galactose units to 1 arabinose unit), galactan (composed of repeating galactose monomers), galactomannan (composed of mannose monomers with galactose side groups) and guar gam (composed of β-D-mannose monomers with every other monomer in the chain having an α-D-galactose residue attached thereto).

The term “drug” as used herein, refers to a therapeutically active compound being preferably oxidation-sensitive. As the drug needs to be attached to the polymer preferably via a covalent bond, said drug is preferably selected amongst hydroxylated (or thiolated) and aminated active compounds. The O (or S) atom of the hydroxylated compound or the N atom of the aminated compound, through which the attachment to the polymer is achieved, may be inherent to the drug or chemically modified thereon in order to facilitate such attachment.

Preferably, the drug is thus selected from polyene antibiotics, low molecular weight drugs having a molecular weight of less than about 2,000 Dalton, high molecular weight drugs having a molecular weight of between about 2,000 and 6,000 Daltons, amine drug derivatives, peptides or polypeptides and analogs thereof.

Non-limiting examples of hydroxylated drugs are dexamethasone, daunorubicin, cytarabine, salicylic acid, santalol, and propanolol. Non-limiting examples of polyene antibiotics are Nystatin and Amphotericin B (AmB).

Non-limiting examples of low molecular weight drugs are 5-amino salicylic acid, aminoglucoside antibiotics, polyene antibiotics, flucytosine, pyrimethamine, sulfadiazine, dapsone, trimethoprim, mitomycins, methotrexate, doxorubicin, daunorubicin, polymyxin B, propanolol, cytarabine and santalol.

The term “amine drug derivatives” refers to oligopeptide esters of hydroxyl containing drugs, which carry a primary amine or have been chemically modified to carry a primary amine. The term “oligopeptide” as used herein, typically refers to a peptide chain comprising 20 amino acids or less, being identical or different. Examples of such derivatives include, but are not limited to, alanyl-Taxol, triglycyl-Taxol, alanyl-glycyl-dexamethasone, glycyl-dexamethasone and alanyl-dexamethasone. The polypeptides are those having a molecular weight of less than about 6,000 Daltons, preferably having one or more oxidizable amino acids such as cysteine, methionine, tyrosine, histidine and tryptophan. Examples of such polypeptides include, but are not limited to, luteinizing hormone releasing hormone (LHRH), bradykinin, vasopressin, oxytocin, somatostatin, thyrotropin releasing factor (TRF), gonadotropin releasing hormone (GnRH), insulin and calcitonine.

The term “polypeptide analogs” refers to chemically modified bioactive polypeptides including cyclic derivatives, N-alkyl derivatives, derivatives in which fatty acids are attached to the amino acid terminals or along the peptide chain, and reverse amino acid derivatives.

As used herein, the expression “C_{polymer}—N_{drug}” refers to the bond between a C atom of the polymer and an N atom on the drug molecule and the expression “C_{polymer}—O_{drug}” refers to the bond between a C atom of the polymer and an O atom of the drug. The N atom of the drug molecule may for example be an amine group (primary or secondary, charged or neutral), amide group or part of a heterocyclic ring system (charged or neutral), and the O atom of the drug may be an hydroxyl group (or hydroxylate) or a carboxylic acid (or carboxylate —O—C(═O)—).

In one embodiment, the C—N bond formed between a C atom of the polymer and an N atom of the drug is a single bond, herein referred to as the “amine bond”. In another embodiment, the C—N bond is a double bond, herein referred to as the “imine bond”.

The conjugate of the invention is said to be substantially free of aldehyde groups if it has at most one aldehyde group, —C(═O)H, (which is capable of imparting toxicity to the polymer) per 10 monomers or monosaccharides, preferably one aldehyde group per 20 monosaccharides, and most preferably 1 aldehyde groups per 100 monosaccharides. The test for the abundance of the aldehyde groups may be selected from a variety of analytical methods known to a person skilled in the art. One exemplary test disclosed hereinafter makes use of the quantitative titration of hydroxylamine hydrochloride.

In another preferred embodiment of the invention, the conjugate of the invention comprises a combination of the following monomers:
(a) at least one monomer of said polymer, e.g. monosaccharide of a polysaccharide;
(b) at least one oxidized form of said monomer (of (a)), being substantially free of aldehyde groups; and
(c) at least one of said oxidized forms (of (b)), being conjugated to a drug, and being substantially free of aldehyde groups;

wherein said combination affords a water-soluble or water dispersible polysaccharide, being substantially free of aldehyde groups.

In one embodiment, said polymer is a polysaccharide and the conjugate of the invention comprises a combination of the following monosaccharides:
(a) at least one monosaccharide of a polysaccharide such as dextran, said monosaccharide being glucose;
(b) at least one oxidized open-ring form of glucose, being substantially free of aldehyde groups; and
(c) at least one of said oxidized open ring form of glucose, being conjugated to a drug, and being substantially free of aldehyde groups;

wherein said combination affords a water-soluble or water dispersible dextran, being substantially free of aldehyde groups.

Preferably, the conjugate of the invention comprises at least one of each of monosaccharides (a) to (c). In one embodiment, the monosaccharide (a) constitutes between about 10 and 98% of the weight of the conjugate. In another case, the oxidized form (b) constitutes between about 10 to 60% of the weight of the conjugate. In yet another embodiment, the drug conjugate (c) comprises between about 1 to 50% of the weight of the conjugate.

The term “monomer” of group (a) above refers within the context of the present invention to a monomer building block of the polymer or preferably the monosaccaride of a polysaccharide. Non-limiting examples of such monosaccharides are glucopyranose (the repeating unit in starch), glucose (the repeating unit in glycogen, dextran and cellulose), maltotriose (the repeating unit in pullulan), β-(1-4)-linked D-glucosamine and N-acetyl-D-glucosamine (the repeating units in chitosan), arabinose and galactose (the repeating unit in arabinogalactan, AG) and galactose (the repeating units in galactan).

The oxidized form (group (b) above) of the monosaccharides is the open ring dialdehyde form resulting from oxidation of the monosaccaride units of the polysaccharide chain. In order to form the substantially aldehyde-free oxidized forms, the open-ring dialdehyde is chemically modified by reacting the free aldehyde groups with agents having reactivity thereto affording a group selected from ethers, esters, amines, amines, amides, acetals or hemiacetals.

The at least one oxidized form of said saccharide, being conjugated to a drug (group (c) above) is of the general Formula I. It should be noted that the structure presented is a general representation of an open-ring monosaccharide which may be different for different polysaccharides or polymers. Thus, the general structure also encompasses different ring sizes, stereoisomers, different substitutions and molecular weights.

In the general Formula I:

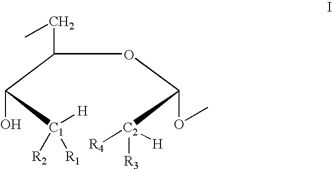

R1 is absent or selected from H, OH and —O-alkyl group;

R2 is a drug (as defined hereinbefore) being conjugated to said monomer via an N or O atom, said conjugation via an N atom may be via a C1-N single or double bond;

when said conjugation is via a C1-N double bond, R1 is absent and the N atom may or may not be further protonated;

when via a C1-N single bond, R1 is H and said N atom may be protonated by one or two hydrogen atoms;

R3 is absent or selected from H, OH, —O-alkyl group, —N-alkyl group, amino acid, lipid, glycolipid, peptide, oligopeptide, polypeptide, protein, glycoprotein, sugar and oligosaccharide;

R4 is absent or selected from a drug (as defined hereinbefore), —O-alkyl group, —N-alkyl group, amino acid, lipid, glycolipid, peptide, oligopeptide, polypeptide, protein, glycoprotein, sugar and oligosaccharide; and

when each of R3 and R4, independently of each other is —O- or N-alkyl group, said alkyl groups together with the O or N atoms to which they are bonded and the C2 atom may form a heterocyclic ring system.

The drug of R2 may or may not be the same as the drug of R4.

The term “amino acid” refers, as may be known to a person skilled in the art, to an organic molecule containing both an amino group and a carboxyl group, including both alpha and beta amino acids. The term “peptide” refers to a short chain of amino acids linked together by peptide bonds in a specific sequence. The term “polypeptide” refers to linear polymers composed of a plurality of amino acids. The term also encompasses proteins.

The term “lipid” refers, as may be known to a person skilled in the art, to an organic molecule that is insoluble in water but tends to dissolve in nonpolar organic solvents. This class also includes the phospholipids. The term “glycolipid” refers to lipid molecules, as defined, with a sugar residue or oligosaccharide attached to the polar headgroup.

The term “sugar” refers to short carbohydrates with a monomer having the general formula (CH₂O)n. Non-limiting examples are the monosaccharides glucose, fructose and mannose, and the disaccharide sucrose. The term “oligosaccharide” refers to a short linear or branched chain of covalently linked sugars.

The term “glycoprotein” refers to any protein with one or more oligosaccharide chains covalently linked to the amino-acid side chains.

In the general Formula I, R4 may be absent and the N atom of the drug bonded to C1 may also be bonded to C2 via a C—N single or double bond, forming a ring structure.

In one embodiment of the general Formula I, the drug being bonded to said polysaccaride is selected from AmB, doxorubicin, mitomycin C, polymyxin B, paclitaxel, gentamicin, dexamethasone, 5-amino salicylic acid, and somatostatin. Preferably, said drug is AmB.

In another embodiment, the monosaccharides are selected from glucose, D-glucosamine, arabinose and galactose or derivatives thereof. In yet another embodiment, said polymer is a homo-polysaccharide, constructed of unoxidized monomers, oxidized monomers and conjugated monomers of the same monosaccharide. In another embodiment, the polysaccharide is a mixed or co-polysaccharide constructed of unoxidized monomers, oxidized monomers and conjugated monomers of at least two different monosaccharides.

In a preferred embodiment, R3 is OH and R4 is a O-alkyl wherein said alkyl is a lower alkyl, i.e. an alkyl having between one and 9 carbon atoms, such as ethyl, or a higher alkyl, i.e. an alkyl having at least 10 carbon atoms, such as cholesterol.

In another preferred embodiment, R3 is OH and R4 is an N-alkyl, bonded to C2 via an amine bond.

In yet another preferred embodiment, R3 is absent and R4 is an N-alkyl, bonded to C2 via an imine bond.

In a further preferred embodiment, R3 is H and R4 is an O-alkyl.

In another preferred embodiment, R3 is OH and R4 is an O-alkyl.

In yet another preferred embodiment, R3 and R4 are each, independently of each other an O-alkyl.

In still another preferred embodiment, R3 is an N-alkyl, bonded to C2 via an amine bond, and R4 is an O-alkyl.

In a still further preferred embodiment, R3 is H and R4 is an N-alkyl, bonded to C2 via an amine bond.

In still another preferred embodiment, R3 and R4, independently of each other are each an N-alkyl, bonded to C2 via an amine bond.

In another embodiment, R3 is absent and R4 is an amino acid bonded to C2 via an imine bond, said amino acid being preferably lysine.

In another embodiment, R3 is H and R4 is an amino acid being preferably lysine.

In yet another embodiment, R3 is absent and R4 is ═NCH₂CH₂OH, wherein the N atom may be neutral or charged.

In still another embodiment, R3 is H and R4 is —NZCH₂CH₂OH, wherein Z may be H or a substituent as defined hereinabove and the N atom may be neutral or charged.

In another embodiment, R3 is OH and R4 is —OCH₂CH₃.

In yet another embodiment, said polymer is dextran, chitosan or arabinogalactan, said drug is AmB and R4 is ═NCH₂CH₂OH or —NZCH₂CH₂OH wherein Z is H or alkyl, —OCH₂CH₃.

The term “alkyl” as used herein refers broadly to a carbon chain of between 1 and 50 carbon atoms. The carbon chain may be substituted or unsubstituted, straight or branched, cyclic or acyclic. Substitution of said alkyl may be by one or more groups or atoms, such as halides (I, Br, Cl and F), heteroatoms (such as N, O, S, P), —OH, —NO₂, —NH₂— aryl, —S(═O)—, —S(═O)₂O—, —C(═O)NH₂—, and others. The term also refers to inner chain alkylene groups having between 1 and 50 carbon atoms and to carbon chains being partially or fully conjugated by C—C double or triple bonds or aromatic moieties. The term “lower alkyl” refers to an alkyl having between one and 9 carbon atoms and the term “higher alkyl” refers to an alkyl having 10 carbon atoms or more.

Non-limiting examples of such alkyl groups are methyl, ethyl, propyl, isopropyl, isobutyl, n-butyl, sec-butyl, tert-butyl, isohexyl, allyl (propenyl), propargyl (propynyl), fluorenyl, phenyl, and naphthyl.

The term “—N-alkyl group” refers to an alkyl group being bonded to the polymer via an N atom which may be a secondary, tertiary or quaternary amine or imine, which may be protonated, alkylated, neutral or charged. The term “—O-alkyl-group” refers to an alkyl group being bonded to the polymer via an O atom.

The substituted or unsubstituted —N-alkyl or —O-alkyl-group, amino acid, lipid, glycolipid, peptide, oligopeptide, polypeptide, protein, glycoprotein, sugar and oligosaccharide of R3 or R4 may be selected from: (i) moieties which substantially have no effect on the biological/therapeutic activity, specificity, chemical and/or physical characteristics of the unmodified conjugate and (ii) moieties which impart to the modified conjugate at least one additional characteristic selected from: hydrophobicity, hydrophilicity, acidity, solubility, dispersability, chemical reactivity, specificity to a target tissue, modified therapeutic activity and affinity towards a certain receptor or biological active site.

Non-limiting examples of moieties which substantially have no effect on the conjugate are derived from ethanolamine, hydroxylamine, propylene glycol, glycerol, and ethanol.

Non-limiting examples of moieties which may impart to the conjugate additional characteristics are: (1) cholesterol and derivatives thereof, which may bestow on the conjugate hydrophobic properties and help a hydrophilic drug to cross hydrophobic barriers; (2) glucosamine, which may increase the hydrophilicity of the conjugate; (3) amino acids such as glycine, alanine, phenylalanine, glutamic acid, aspartic acid or short oligopeptides thereof which may be used to increase the acidity of the conjugate; (4) amino acids such as lysine, ornythine or oligopeptides thereof which may be used to decrease the acitidy of the conjugate; (5) bifunctional molecules such as lysine, spermine, spermidine and other non-toxic diamines which may be used for crosslinking or branching of the conjugate; and (6) hydrophobic molecules such as the fatty acid amines: stearyl amine, oleyl amine, and palmitoyl amine which may be used to increase the lipophilicity of the conjugate.

In one embodiment, said moiety is capable of imparting to the conjugate the required hydrophobicity so that the resulting modified conjugate of the invention becomes insoluble in water and thus may be suitable for the preparation of nanoparticles, liposomes, micellar dispersions, and colloidal dispersions. In another embodiment, such modified conjugate is used to coat lipophilic surfaces.

In another aspect of the present invention, there is provided the use of any one of the conjugates of the present invention for the preparation of a composition. Preferably, said composition is for pharmaceutical applications.

In one embodiment, there is provided the use of a conjugate of the invention for the preparation of a pharmaceutical composition effective as an antibiotic.

In another embodiment, there is provided the use of a conjugate of the invention for the preparation of a pharmaceutical composition effective as an antiparasitic.

In another embodiment, there is provided the use of a conjugate of the invention for the preparation of a pharmaceutical composition effective as an anticancer.

In another aspect of the present invention, there is provided a composition comprising at least one conjugate of the present invention. Preferably, said composition comprises also a carrier or an inactive ingredient. More preferably, said composition is a pharmaceutical composition and said carrier is a pharmaceutically acceptable carrier.

The pharmaceutically acceptable carriers may for example be selected from vehicles, adjuvants, excipients, or diluents, as is well-known to those who are skilled in the art. It is preferred that the pharmaceutically acceptable carrier be one which is chemically inert to the drug and the conjugate as a whole and one which has no detrimental side effects or toxicity under the conditions of use.

The choice of carrier will be determined in part by the particular conjugate, as well as by the particular application. The conjugates of the invention or any composition comprising thereof may be made into formulations for oral, aerosol, parenteral, subcutaneous, intravenous, intramuscular, interperitoneal, rectal, and vaginal administrations.

Additionally, the conjugates of the present invention may be made into hydrogels, preferably biodegradable, and thus be formulated for injection, coating on stents or in situ implantation. The conjugates of the invention may also be made into nanoparticles, micellar dispersions, liposomes and modified release formulation which utilizes the various drug release properties of the conjugates.

The pharmaceutical composition of the invention may be used for the treatment of any one disease or disorder treatable by any one drug employed in the conjugates as defined herein. For example, the conjugates may be used as antibiotics, antiparasitic or anticancer agents in a treatment of a subject, human or non-human, in need thereof.

In this respect, the term “treatment” or any lingual variation thereof refers to the administering of a therapeutic amount of the composition of the present invention which is effective to ameliorate undesired symptoms associated with a disease, to prevent the manifestation of such symptoms before they occur, to slow down the progression of the disease, slow down the deterioration of symptoms, to enhance the onset of remission period, slow down the irreversible damage caused in the progressive chronic stage of the disease, to delay the onset of said progressive stage, to lessen the severity or cure the disease, to improve survival rate or more rapid recovery, or to prevent the disease form occurring or a combination of two or more of the above.

The composition of the invention may be administered in any suitable formulation, alone or in combination with other known treatments, i.e. chemotherapy.

In another aspect of the present invention, there is provided a method for the preparation of a conjugate according to the invention, the method comprising:

(a) providing an unmodified water-soluble conjugate of a polymer, i.e. polysaccharide and a drug, said polysaccharide having at least one aldehyde group, said drug being conjugated to said polysaccharide via a bond selected from an imine (—C_{polymer}═N_{drug}—), amine (—C_{polymer}—N_{drug}R—), amide (—C_{polymer}—N_{drug}C(═O)—), ether (—C_{polymer}—O_{drug}—) and carboxyl (—C_{polymer}—O_{drug}—C(═O)—) bonds; and

(b) reacting said unmodified conjugate with an agent having reactivity towards said aldehyde group, as disclosed hereinabove, and substantially no reactivity or low reactivity towards the drug or said bond; said agent preferably having a molecular weight lower than 500 Dalton, more preferably less then 200 Dalton; thereby obtaining a conjugate substantially free of aldehyde groups.

Optionally, the method further comprises the step of reducing the imine bond between the drug and the polysaccharide.

In one embodiment, step (a) and step (b) are performed in sequence. In another embodiment, the method is employed as a one-pot reaction as may be known to a person of skill in the art of organic synthesis.

In a preferred embodiment, the resulting conjugate, substantially free of aldehyde groups, has a reduced toxicity relative to the unmodified conjugate of step (a) above.

In another embodiment, the unmodified conjugates of method step (a) are selected amongst the conjugates disclosed in U.S. Pat. Nos. 5,567,685 and 6,011,008.

It is to be understood that the conjugates of the present invention may contain chiral centers. Such chiral centers may be of either the (R) or (S) configuration, or may be a mixture thereof. Thus, the conjugates provided herein may be enantiomerically pure, or be stereoisomeric or diastereomeric mixtures. In the case of amino acid residues, such residues may be of either the L- or D-form. It is to be understood that the chiral centers of the conjugates may undergo epimerization under certain conditions.

In still another aspect of the present invention, there is provided a conjugate obtained by the preparative method of the invention.

In yet another aspect, there is provided a conjugate obtainable by the preparative method of the invention.

In still another aspect, there is provided a conjugate prepared by reacting an unmodified conjugate having a plurality of aldehyde groups with a reagent capable of chemically transforming, as may be known to a person skilled in the art, each of said plurality of aldehyde groups into a group selected from amine, imine, amide, acetal, hemiacetal, ether and ester. For aldehyde group transformations, see for example Comprehensive Organic Transformations: A Guide to Functional Group Preparations, R. C. Larock, Wiley-VCH; 2 Ed. 1999.

In yet another aspect of the present invention, there is provided a method for the reduction of the toxicity associated with the unmodified conjugates, such as those disclosed in U.S. Pat. Nos. 5,567,685 and 6,011,008, said method comprises transforming the plurality of aldehyde groups of said unmodified conjugates into a plurality of groups selected from acetals, hemiacetals, amines, and imines.

In one embodiment of the present aspect, the unmodified conjugate is reacted with a polyamine in such a way that said aldehyde groups of the unmodified conjugate are reacted with the amine groups of said polyamine, thus cross linking said conjugate and said polyamine and affording a hydrogel. Preferably, said hydrogel is substantially free of aldehyde groups.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 demonstartes the cytotoxicity of a dextran polyaldehyde. The cytotoxicity test was performed using the ³H-thymidin incorporation method in murine RAW 264.7 cells, by application of dextran (40 kDa) with different degrees of oxidation. Each test was performed twice in triplicate. Mean and standard deviations are shown. The aldehyde concentration was calculated as [2(dose weight,g)×(% degree of oxidation)/(saccharide unit weight, 160 g/mol) mL].

FIG. 2 demonstrates the cytotoxicity of modified dextran polyaldehyde of the invention. The cytotoxicity test was performed using the ³H-thymidin incorporation method in murine RAW 264.7 cells, by application of dextran (40 kDa). Each test was performed twice in triplicate.

FIG. 3 demonstrates the in vitro cytotoxicity of dextran-AmB (imine) and dextran-AmB-ethanolamine conjugates. The cytotoxicity test was performed by the ³H-thymidin incorporation method in murine RAW 264.7 cells. Conjugates were applied with the same amount of drug. Each experiment was performed twice in triplicate.

FIG. 4 shows AmB release from dextran-AmB conjugates in solution at 37° C. AmB release was evaluated by HPLC. Each data point is an average of two different batches.

### DETAILED DESCRIPTION OF EXPERIMENTAL RESULTS

A person of skill in the art would recognize that the examples provided herein are presented as non-limiting embodiments of the present invention. The Schemes and the open-ring structure shown herein for the monosaccharide having the general structure of Formula I are intended as general representations of a polysaccharide or a monosaccharide and should not be regarded as the claimed structure of the monomer or as reciting a preferred embodiment. This general structure of Formula I or any such structure shown in the Schemes may be substituted or be of a different ring size as may be characteristic of other polymers or polysaccharides. Thus, a person skilled in the art would be of the knowledge to replace one polysaccharide under another employing the necessary modifications.

### Example 1

### Synthesis of Dextran Polyaldehyde

Dextran having MW of above 40,000 was oxidized with different amounts of periodate to form a range of oxidized dextrans with different aldehyde content (Scheme 1). Dextran polyaldehyde with a degree of oxidation between 1.5% and 50% (1.5%, 5%, 8%, 15%, 25%, and 50%) was prepared in an aqueous solution by the addition of controlled amounts of potassium periodate (0.0836, 0.2875, 0.46, 0.8625, 1.4375, and 2.875 g, respectively) to 1 g of dextran and stirred in a light-protected container at room temperature for 6 h. The resulting polyaldehydes were purified from iodate and unreacted periodate ions by Dowex-1 anion-exchange chromatography (acetate form, pH 7). Dowex acetate was obtained by pretreatment of the commercial anion exchanger with aqueous 1 M acetic acid. The purified oxidized dextran solution was dialyzed through 3500 molecular weight cutoff dialysis tubing (Membrane Filtration Products Inc., San Antonio, Tex.) against double distilled water (DDW) (5 L changed 4 times) for 48 h at 4° C. and then lyophilized for 24 h to dryness.

Determination of the degree of oxidation was performed as follows: oxidized dextran (0.1 g, 0.625 mmol) was dissolved in 25 mL of 0.25 M hydroxylamine hydrochloride solution, pH 4.0. The solution was stirred for 3 h at room temperature and then titrated with 0.1 M NaOH standard solution. The titration end point was calculated from the graph describing the change in pH per volume (dpH/dV) versus the titration volume (V). Molecular weight was determined by GPC. Samples at a concentration of 10 mg/mL were eluted with 0.05 M sodium nitrate in DDW through a Shodex (KB-803) column at a flow rate of 1 mL/min. The molecular masses of the eluted samples were estimated by use of pullulan standards in the range of 5,000-110,000 Da (PSS, Mainz, Germany).

Results: There was a linear correlation between the amount of potassium periodate used for oxidation and the aldehyde content of the oxidized dextran. The degree of oxidation of dextran, after reaction with different molar ratios of periodate (1:1, 1:2, 1:3, 1:5, 1:10, and 1:33 periodate:saccharide units), and the molecular weights of the oxidized dextrans are summarized in Table 1.

TABLE 1
Characterization of dextrans after oxidation with
different molar ratios of KIO₄.

| KIO₄/saccharide units | Aldehyde | MW | Polydispersity |
|---|---|---|---|
| (mole ratio) | content, %^{a} | (GPC)^{b} | (MW/Mₙ) |
| 1:1 | 52 | 32 019 | 2.39 |
| 1:2 | 25 | 30 520 | 1.59 |
| 1:3 | 15 | 31 787 | 1.56 |
| 1:5 | 8 | 32 356 | 1.57 |
| 1:10 | 5 | 30 491 | 1.58 |
| 1:33 | 1.5 | 31 342 | 1.56 |

In Table 1:
^{a}Degree of oxidation was determined by the hydroxylamine hydrochloride method. Percent of oxidation is the percent of saccharide units oxidized to yield two aldehydes per unit;
^{b}Molecular weight was determined by gel-permeation chromatography.

All oxidized dextrans had a similar average MW of about 32,000 and polydispersity of about 1.6. There was a slight increase in polydespersity value for the highly oxidized dextran (P=2.39), which is related to the large excess of periodate used for oxidation.

### Example 2

### Synthesis of Modified Dextran

Reduced Dextran—Oxidized dextran (1 g, 50% oxidation) was dissolved in 100 mL of DDW. NaBH₄ (1 g) was added and the reaction mixture was stirred for 24 h. The solution was purified by dialysis and lyophilized (as described in Example 1 above).

Dextran Acetal—Oxidized dextran (1 g, 50% oxidation) was dissolved in 100 mL of ethanol and stirred for 24 h. Dextran acetal was precipitated in DDW and lyophilized (as described in Example 1 above).

Dextran-Ethanolamine Imine/amine—Dextran (2 g, 50% oxidation) was dissolved in 200 mL of borate buffer, pH 11, and 0.41 mL (1.1 mol equiv) of ethanolamine was added. The reaction mixture was stirred for 24 h, after which a sample of 100 mL was removed, purified by dialysis, and lyophilized to dryness (as described in Example 1 above) to obtain the imine form. To obtain the amine form, 1 g of NaBH₄ was added to the remaining 100 mL of reaction solution. The reaction mixture was stirred for 24 h, purified by dialysis, and lyophilized (Scheme 1).

### Example 3

### Synthesis of Dextran-Amphotericin B (AmB) Imine/Amine Conjugate

In the first step, oxidized dextran (50% oxidation) was prepared, followed by a second step of conjugation of the oxidized dextran to AmB (see Scheme 2). In a typical experiment, 1 g of oxidized dextran with a degree of oxidation of 50% of the saccharide units was dissolved in 100 mL of borate buffer, pH=11. AmB powder (0.25 g) was added, and the mixture was stirred at room temperature in a light-protected container for 48 h. The pH of the reaction mixture was maintained at 11 during the reaction. A clear yellow-orange solution of the imine conjugate was obtained, purified by dialysis, and lyophilized for 24 h (as described in Example 1). The amine conjugate was obtained by addition of NaBH₄ to the imine conjugate reaction mixture and continuation of the reaction overnight. During the reduction process, a change of color from yellow-orange to light yellow was observed. The amine conjugate was purified by dialysis and lyophilized (as described in Example 1).

Dextran-AmB-ethanolamine (imine) conjugate was prepared, as shown in Scheme 2, by adding (1.1 mol equiv of aldehyde content) of ethanolamine to the imine conjugate mixture and continuing the reaction overnight. The pH of the reaction was maintained at 11. The dextran-AmB-ethanolamine conjugate was purified by dialysis and lyophilized to dryness (as described in Example 1).

### Example 4

### Measurement of AmB Content in Conjugates

AmB content in the conjugates of the invention was determined by UV absorbance at 410 nm, by use of dextran-AmB conjugates with known amount of drug as standards. Purity of the conjugates was determined by HPLC on a C18 reverse phase column (LichroCart 250-4, Lichrospher 100, 5 μm). A mixture of 70% acetonitrile/27% water/3% acetic acid at a flow rate of 1.8 mL/min was used as eluent. UV detection was at 410 nm. For both tests the conjugate samples were prepared at a concentration of 0.3 mg/mL in DDW.

### Example 5

### Synthesis of Arabinogalactan (AG)-Lysine Conjugates

AG with an average molecular weight of 20,000 Da (1 g, 0.006 mol) was dissolved in 20 ml of double distilled water (DDW), followed by the addition of potassium periodate (1.4 g, 0.006 mol), and the reaction mixture was stirred at room temperature for 4 h for complete dissolution of the oxidizer. The oxidized AG thus obtained, was separated from excess periodate and reaction by-products in a column filled with Dowex-1 in the acetate form. The purified oxidized AG solution was then dialyzed through a dialysis tubing (12,000 Da molecular weight cutoff) against DDW (5 liters×4) for 48 h at 4° C., and lyophilized to dryness. Alternatively, the conjugate was purified by ultrafiltration using a 5,000 molecular weight cutoff filter until a pure conjugate was obtained.

The degree of oxidation was determined by reacting the conjugate with hydroxylamine hydrochloride and titrating the formed free HCl with NaOH solution to the end point of phenol phthalein. AG with a degree of oxidation of 0.005 mol aldehydes per 1 g polysaccharide was dissolved in 0.1 M carbonate buffer pH 8.5 (10 ml), followed by the addition of lysine hydrochloride (1% w/w, 10 mg), and the reaction mixture was shaken at 37° C. for 24 h. The imine conjugate gel was divided in two; one portion was reacted with excess ethanolamine to block the extra aldehyde groups. After 5 hours the gel was separated and washed carefully to remove unreacted ethanolamine and other small molecules. The other half of the original gel portion and half of the ethanolamine derivative portion were reduced to the amine form by the addition of sodium borohydride (1.1 moles NaBH₄/mol of saccharide unit in AG) to the reaction mixture for 12 h at room temperature, and then drying under vacuum.

### Example 6

### Dextran Polyaldehyde In Vitro Toxicity

Serial dilutions of dextrans with different degrees of oxidation (1.5%-50% oxidation) were prepared in RPMI 1640 growth medium. The final aldehyde concentrations in the test were 0.01-34 μmol/mL. Oxidized dextran toxicity was compared to glutaric polyaldehyde toxicity, which was added in concentrations between 0.15 and 4.12 μmol/mL aldehyde groups.

The cytotoxicity of dextran derivatives was evaluated in murine RAW 264.7 cells, an internationally recognized cell line for examination of drug effects.

Growth inhibition was estimated by the ³H-thymidine incorporation method. Cells were cultured in flat-bottom flasks at 37° C. Before each experiment the cells were washed and removed by trypsin treatment or scraped from the flask bottom, and an appropriate volume was centrifuged, resuspended, and diluted in growth medium to the desired cell concentration. The growth medium consisted of RPMI 1640 and 10% fetal calf serum (FCS). By use of an automated dispenser, 200 μL of cell suspension was added to each well of a microtiter plate. After incubation overnight, the appropriate drug concentration, in triplicate, was added to test wells. Drug-free medium was used as control. ³H-Thymidine (0.5 μCi) in 20 μL of medium was added the next day, and the plate was harvested and read by liquid scintillation counter (LKB, Finland) after an additional 24 h. The percent growth inhibition of the cells by the drug tested was calculated as [100−(count with drug/control count)×100]. The IC₅₀ of the drugs, defined as the concentration that inhibits 50% of the incorporation, was determined graphically from inhibition of incorporation curves.

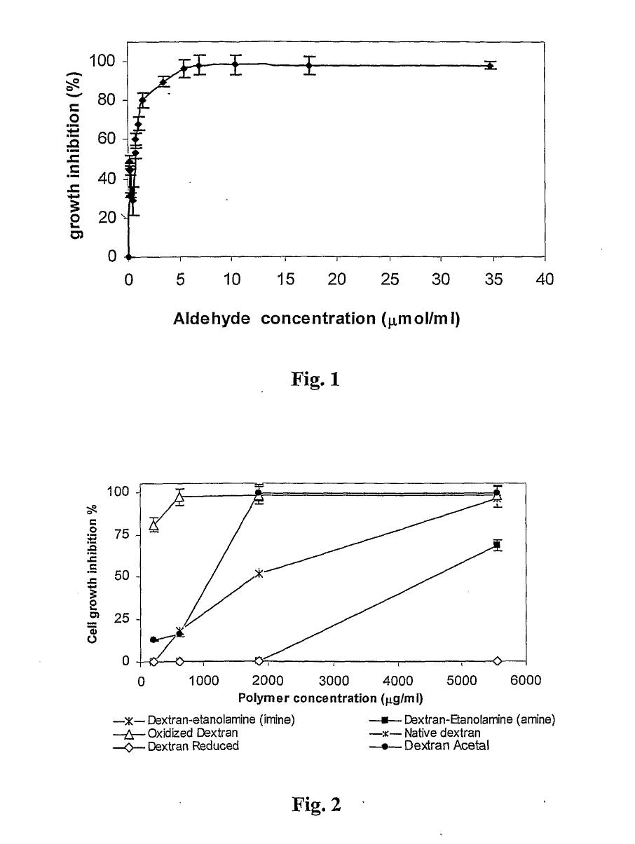

Results: The cytotoxicity experiment was performed by incubating the cells with the same amounts of the oxidized dextrans. A correlation between the aldehyde content in the oxidized dextrans and cell growth inhibition was found (FIG. 1). The presence of aldehyde groups caused cytotoxicity, with an IC₅₀ of 3 μmol/mL. Exposure of the cells to aldehyde concentration higher than 7 μmol/mL caused complete inhibition.

### Example 7

### Cytotoxicity Evaluation of Modified Dextran Polyaldehyde

The purpose of this experiment was to confirm that the cell growth inhibition described previously was caused only by the aldehyde groups. Therefore, the aldehyde groups were chemically transformed to nontoxic groups such as a hydroxyl (end group of ethanolamine) or aliphatic groups (end group after reaction with ethanol). All modifications were made on dextran polyaldehyde with the highest degree of oxidation (50%) (Scheme 1).

Serial dilutions of oxidized dextran and modified dextran were prepared in RPMI 1640 broth medium. The final dextran concentration in the test ranged from 44 to 5555 μg/mL.

To establish that the aldehyde groups were primarily responsible for cytotoxicity, native dextran and dextran with completely eliminated aldehydes (by reduction to hydroxyl) were evaluated. Dextran with 50% oxidation was used as a positive toxicity control. Drug effect and the IC₅₀ were defined as previously described (Example 6).

Results: The toxicity of the modified dextran was evaluated in the cell system disclosed in Example 6. Oxidized dextran caused almost complete growth inhibition at the lowest tested concentration (130 μg/mL). Modification with ethanol to form hemiacetals substantially reduced the toxicity of the polymer, with a complete growth inhibition observed at concentration of the dextran hemiacetal higher than 1800 μg/mL. Modification with ethanolamine (imine form) reduced the toxicity by 16-fold, and an additional reduction step to form dextran-ethanolamine (amine) further reduced the toxicity relative to that of the unmodified dextran. As may be noted from Table 2, the conjugate of dextran and ethanolamine (prepared according to the procedure of Example 2 above) exhibited a considerable reduction in toxicity, from IC₅₀=130 to 2000 μg/mL. Moreover, reduction of the imine bond to the amine bond, further improved the toxicity to IC₅₀=4500 μg/mL (35-fold).

The complete elimination of aldehydes, e.g. by reduction of the aldehyde groups of oxidized dextran (herein referred to in Table 2 as the reduced dextran) entirely prevented the toxicity in the tested dose range. A similar effect was observed in the native dextran. For easier comparison of the results, IC₅₀ values were graphically estimated as shown in FIG. 2 and summarized in Table 2.

TABLE 2
In vitro cytotoxicity of modified dextran as compared
to oxidized dextran (50%) and glutaraldehyde.

| Compound | IC₅₀ (μg/ml)^{a} |
|---|---|
| Native Dextran | >5000 |
| Dextran Reduced | >5000 |
| Dextran-Ethanolamine (imine) | 2000 |
| Dextran-Ethanolamine (amine) | 4500 |
| Dextran Hemiacetal | 1000 |
| Oxidized Dextran | 130 |
| Glutaraldehyde | <0.15 |

^{a}IC₅₀ values were determined from in vitro cytotoxicity experiments. The cytotoxicity test for different modifications of dextran 40 kDa was performed by the measurement of ³H-thymidine incorporation in RAW 264.7 cells. The cytotoxicity was compared to the effect of native dextran and oxidized dextran.

### Example 8

### Dextran-AmB Conjugates In Vitro Toxicity

The cytotoxicity test for the conjugates was performed in the same cell system as previously described (Example 7). Conjugates were prepared in the concentration range in which the oxidized dextran had exhibited cytotoxicity.

Results: After synthesis, the purity of the conjugates was evaluated by HPLC as described in Example 4. The HPLC showed the presence of fully bound drug conjugates. No free drug was detected. The toxicity was thus assumed to stem from the conjugate itself and not from free unconjugated drug molecules.

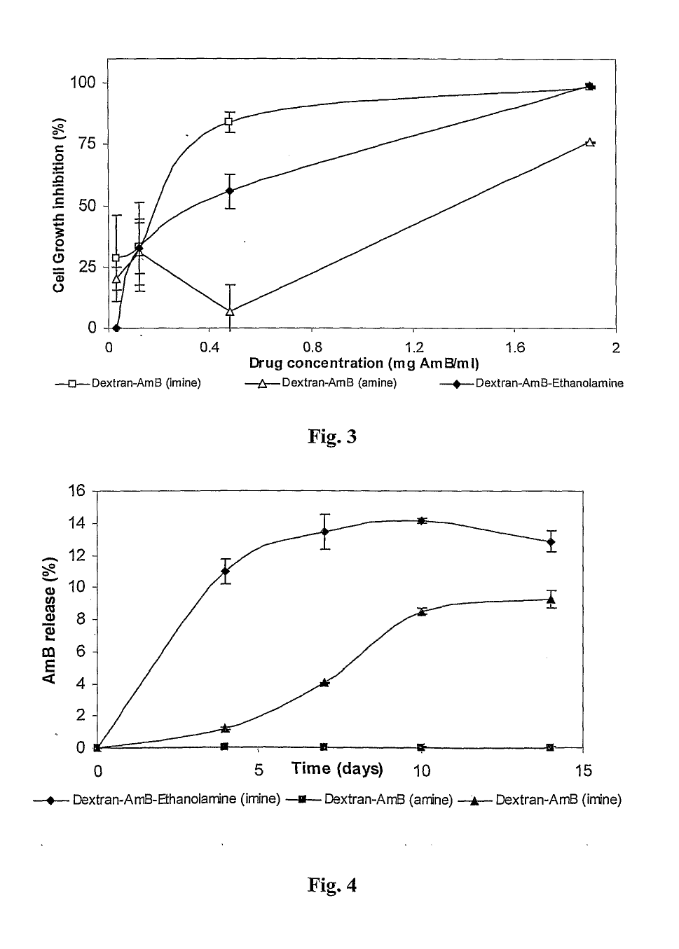

The toxicity was evaluated in comparison with dextran-AmB imine conjugate (previously described in U.S. Pat. No. 5,567,685 mentioned hereinabove). The AmB concentration was similar in all conjugates in order to eliminate the drug influence on conjugate toxicity. AmB-dextran imine conjugates with or without ethanolamine were compared to the AmB-dextran amine conjugate, all containing equivalent AmB amounts, to evaluate the contribution of the remaining aldehyde groups to conjugate toxicity (FIG. 3). Drug effect and the IC₅₀ were defined as previously described.

The IC₅₀ values are summarized in Table 3. Free AmB was extremely toxic to both parasites and cells. As may be noted, the amine and imine conjugates were substantially less toxic than the free AmB but retained a certain degree of toxicity which is believed to stem from the remaining aldehyde groups. The amine conjugate of AmB was least toxic to both the parasites and cells. Without wishing to be bound by theory, the difference in cytotoxicity and antiparasitic activity demonstrated seems to arise from the possible release of the AmB from the imine conjugate after hydrolysis of the imine bond. The release of the drug from the amine conjugate under identical conditions seemed less likely to occur.

Modifying either the imine or amine conjugates with ethanolamine thereby obtaining a substantially aldehyde free conjugate further reduced the toxicity of the conjugate while retaining the activity of the conjugate.

TABLE 3
In vitro activity against Leishmania donovani,
cytotoxicity and hemolysis of conjugates.

| | | Antiparasitic | | |
|---|---|---|---|---|
| | AmB | activity^{a} | Toxicity^{b} | |
| | content | IC₅₀ | IC₅₀ | Hemolysis^{c} |
| Compound | (% w/w) | (μgAmB/ml) | (μgAmB/ml) | (μgAmB/ml) |
| Free AmB | 100 | 0.05 | 9 | 16 |
| Dextran-AmB | 34.4 | 1.2 | 1400 | >500 |
| (amine) | | | | |
| Dextran-AmB | 36.6 | 0.3 | 200 | 250 |
| (imine) | | | | |
| Dextran-AmB - | 32.9 | 0.25 | 400 | >500 |
| Ethanolamine | | | | |
| (imine) | | | | |

^{a}IC₅₀ values were derived from the activity test of AmB and different dextran-AmB conjugates against Leishmania donovani. Parasite growth inhibition was estimated using the ³H-thymidine incorporation method.
^{b}IC₅₀ values were derived from the cytotoxicity test of AmB and different dextran-AmB conjugates against the murine RAW 264.7 cell line. Cell growth inhibition was estimated using the ³H-thymidine incorporation method.
^{c}Hemolysis was evaluated visually after 1 h incubation at 37° C. with Sheep erythrocytes.

### Example 9

### In Vitro Activity Against Leishmania donovani

The in vitro antiparasitic activity was evaluated against Leishmania donovani IS promastigotes. This strain, isolated from a patient in Sudan, was received from the International Reference Center of the Kuvin Center for Infectious Diseases in the Hebrew University of Jerusalem.

Serial dilutions of the tested agents were prepared in RPMI 1640 growth medium. The final AmB concentration in the test ranged from 0.2 to 6 μg/mL. Wells containing drug-free medium served as control. The growth inhibition was estimated by the ³H-thymidine incorporation method. Briefly, 96-well plates were seeded with 60,000 promastigotes/well in 200 μL of medium, and test solutions were added 3 h later. After 24 h of incubation, 0.5 μCi/well ³H-thymidine (in 10% FCS medium) was added, and the cultures were harvested after an additional 24 h. During the experiment the cells were incubated at 25° C. in air. The drug effect and the IC₅₀ of the conjugates were estimated as described before (Example 7).

Results: Both imine conjugates (namely without ethanolamine or conjugated therewith) showed higher activity against Leishmania donovani parasites relative to the amine conjugates, with an IC₅₀ of about 0.3 μg/mL compared to 1.2 μg/mL (Table 3). Without wishing to be bound by theory, this result seems to further support the possible hydrolytic degradation of the imine bond discussed above.

### Example 10

### Doxorubicin-dextran Ethanolamine Imine Conjugate

Doxorubicin (DOX, also adriamycin) was conjugated to oxidized dextran under various reaction conditions. In a typical experiment, 20.0 ml of purified DAD solution (25 mg/ml, MW=19,000) was mixed with an equal volume of 0.2 M borate buffer solution pH 9.1, and 200.0 mg of DOX was added to the polymer solution (10 mg/ml). The pH of the mixture was maintained at pH 8.9±0.1 for 16 h at 37° C. After 16 hours, ethanolamine was added in access and reacted for 5 hours under similar conditions to block the remaining aldehyde bonds. The crude conjugate was dialyzed against DDW for 30 h at 4° C. using molecular porous membrane tubing with a MW cutoff of 12,000, followed by centrifugation for 10 min at 2,000 rpm and lyophilization. The lyophilized light-yellow product (605 mg, 85% yield) contained about 20% of DOX as evaluated by UV absorption at 480 nm.

The lyophilized light-yellow product was stored in a glass container protected from light and air. The release of DOX from the conjugate was determined using dialysis tubing with a pore size of 10,000 cut off. About 10% of the drug was released after 30 hours. In vitro cell culture was conducted to determine the activity of the conjugate. This imine derivative of DOX was effective to the same order of magnitude as the free drug.

### Example 11

### Mitomycin C-Arabinogalactan Glucosamine Imine Conjugate

One gram of arabinogalactan (AG, molecular weight of 28,000) was dissolved in 50 ml solution containing 0.3 g of potassium periodate. The solution was mixed for 3 hours at room temperature. The solution was then passed through a Dowex column and dialyzed and lyophilized to yield a white powder free of oxidizing agent. The pure dialdehyde AG (200 mg) was dissolved in 10 ml boric acid buffer pH 8.9 and mixed with 20 mg of Mitomycin C in 5 ml of water. The solution was mixed for 24 hours. Next glucosamine was added in access and the reaction continued for another 5 hours before the product was purified by ultrafiltration against water and lyophilized to yield the Schiff base.

The amount of conjugated drug was 8% by weight as determined by UV absorption at 280 nm. The molecular weight of the lyophilized product was 26,000 Dalton. The Mitomycin release into the solution and the toxicity were measured as described above in Example 7. The amount of drug found in the solution was about 10% of the total dose after 48 hours at 37° C. in buffer (pH 7.4) solution. The conjugate showed similar anti-cancer activity as compared with the activity of the free drug. The conjugate modified with glucosamine was much less toxic to cells as compared with the same unmodified conjugate.

### Example 12

### Polymyxin B-Arabinogalactan Conjugate

Pure oxidized AG was prepared as described above. The pure dialdehyde AG (200 mg) was dissolved in 10 ml sodium borate buffer pH 8.9 and mixed with 20 mg of Polymyxin B in 5 ml of water. The solution was allowed to mix for 24 hours. The solution was dialyzed with water and lyophilized to yield the Schiff base.

The modified conjugates of AG and polymyxin B were prepared by reacting the Schiff base with such reagents as glucosamine and ethanolamine.

### Example 13

### Paclitaxel-Arabinogalactan Hemiacetal Conjugate

Paclitaxel was reacted with pure oxidized AG at a 1:4 molar ratio of paclitaxel:aldehyde groups in the polymer sample. The reaction was carried out in a mixture of 1:9 DMSO:water solution at pH 8.5 for 8 hours at room temperature. The almost clear solution was treated with excess propylene glycol and was left to react for 5 hours before centrifugation to remove insoluble particles and then lyophilized to yield an off-white powder. The hemiacetal powder was soluble in saline and contained about 8% by weight of the drug as determined by H-NMR.

### Example 14

### Gentamicin-Arabinogalactan Conjugate

The aminoglucoside antibiotic, gentamicin, a water soluble molecule with five amino groups was conjugated to AG via a Schiff base using a procedure similar to that described for amphotericin B. The motivation for this conjugation was to reduce the significant organ toxicity of the drug which limits its use despite its broad range antibacterial activity.

The antimicrobial activity of these conjugates was determined as follows: Saline solutions of equivalent amounts of the drug in free form or the imine AG conjugate were absorbed onto a circular filter paper (6 mm in diameter) and placed on a seeded agar plate with Staphylococcus Aureus (10⁵/ml) and E. Coli incubated for 24 hours at 37° C. Both samples showed an inhibition zone. The free drug showed a large inhibition zone (>20 mm) while the conjugate showed a limited zone (5 mm). The reason for the difference can be explained by the size of the conjugate which has limited diffusion in agar media.

The in vitro toxicity of the conjugate against cells was significantly decreased as compared with the toxicity of the free drug.

In vivo toxicity in mice was determined by inspecting the kidneys of the scarified mice 7 days after injection. The kidneys of mice treated with the conjugate exhibited no signs of drug imparted toxicity as was the case of the control group which was injected with the free drug.

### Example 15

### Dexamethasone-Arabinogalactan Hemiacetal Conjugate

Dexamethasone (10 mg), a poorly soluble anti-inflammatory drug, was reacted with pure 32% oxidized arabinogalactan (100 mg) in borate buffer solution pH 8.9 at room temperature for 24 hours. To the mixture, propylene glycol was added and the reaction continued for 5 hours at which point the solution was lyophilized to yield the hemiacetal conjugate as determined by H-NMR.

### Example 16

### 5-amino Salicylic Acid-Arabinogalactan Glycine Conjugate

5-Amino salicylic acid was conjugated to oxidized AG by reacting 100 mg of 5-amino salicylic acid with 300 mg 32% oxidized AG (MW=19,000) in borate buffer pH 8.9 at room temperature for 24 hours. Glycine was added to the solution and the reaction was continued for 10 hours before purification by ultrafiltration. The imine derivative was obtained in good yields.

In vitro release of the conjugated drug in phosphate buffer pH 7.4 using the dialysis tubing method showed about 10% release after 8 hours at 37° C. The conjugate was much less toxic to cells as compared with the free drug.

### Example 17

### Somatostatin-Arabinogalactan Ethanolamine Conjugate

Somatostatin, a water-soluble peptide drug was conjugated to oxidized AG via an amine bond as follows: to a solution of pure 32% oxidized AG (100 mg in 10 ml borate buffer solution pH 8.9) was added 20 mg of somatostatin and the mixture was stirred over night at 4° C. The clear solution was reacted with excess ethanolamine for 10 hours before purified by ultrafiltration using 10,000 MW cut-off and washed with water to remove the salts and unbound drug. Thereafter, the solution was lyophilized to yield 115 mg of a white solid which corresponded to about 70% binding. The conjugation yield was confirmed by nitrogen analysis of the product.

About 10% of the conjugated drug was released after 12 hours in a buffer at pH7.4 at 37° C. The released drug showed similar UV spectra to the original drug and had the same retention time by HPLC analysis (C18, acetonitrile:water 1:1, 1 ml/min, Rt=5.2 min).

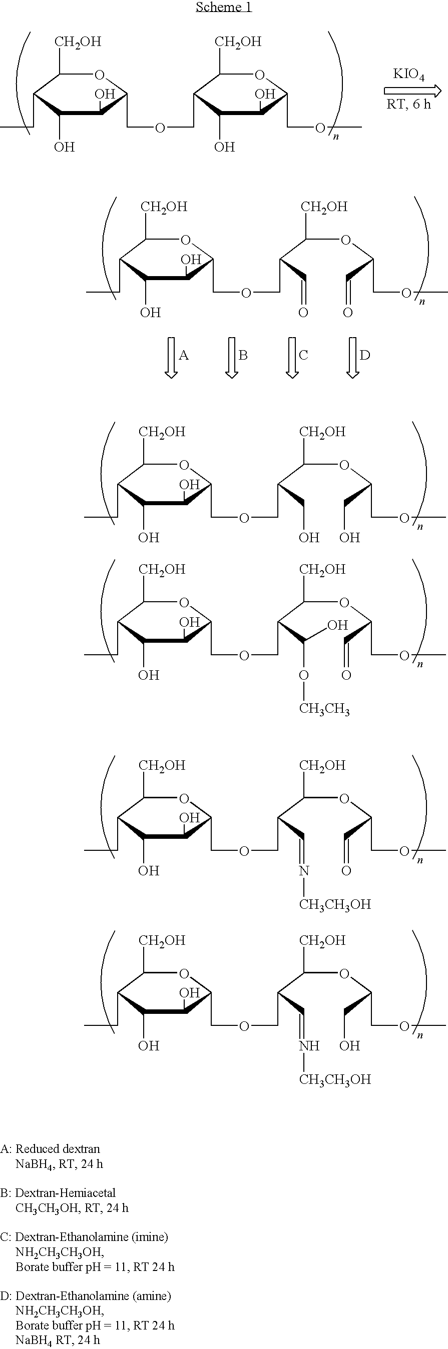

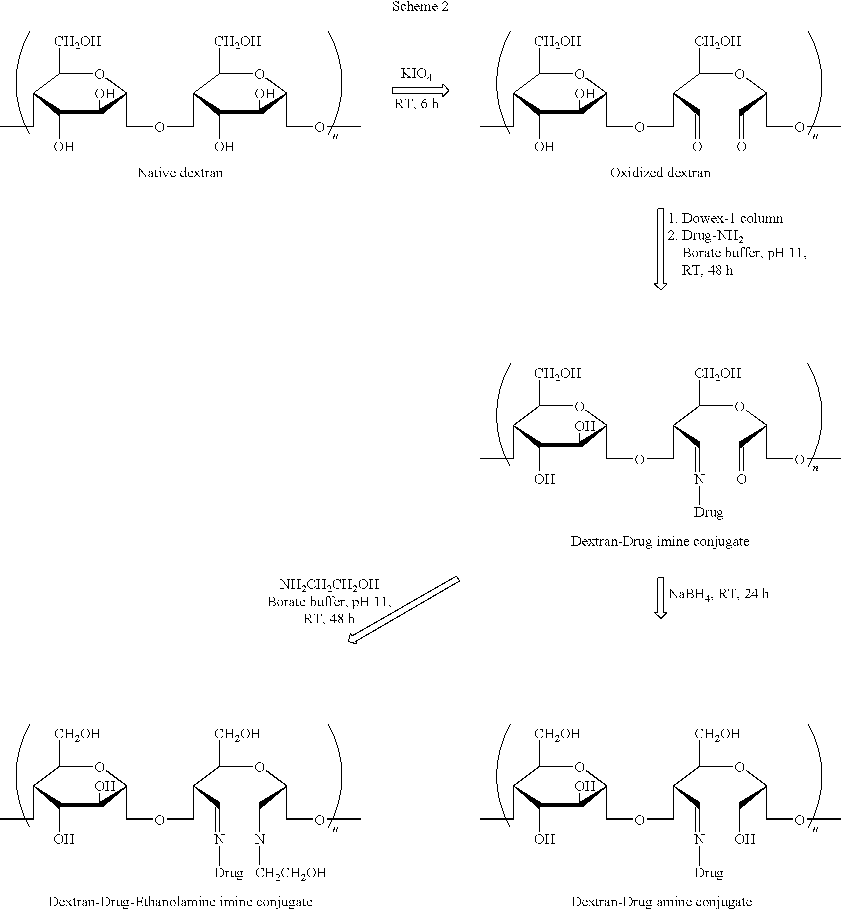

1-71. (canceled)

72. A conjugate of a polymer and a drug, said conjugate comprising a combination of:
(a) at least one monomer of said polymer;
(b) at least one oxidized form of said monomer, said oxidized form being substantially free of aldehyde groups; and
(c) at least one conjugate of said oxidized form with a drug, wherein said conjugate is of the general Formula I, 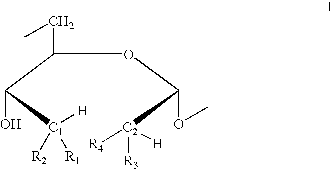
wherein
R1 is absent or selected from H, OH and —O-alkyl group;
R2 is a drug (as defined hereinbefore) being conjugated to said monomer via an N or O atom, said conjugation via an N atom may be via a C1-N single or double bond;
when said conjugation is via a C1-N double bond, R1 is absent and the N atom may or may not be further protonated;
when via a C1-N single bond, R1 is H and said N atom may be protonated by one or two hydrogen atoms;
R3 is absent or selected from H, OH, —O-alkyl group, —N-alkyl group, amino acid, lipid, glycolipid, peptide, oligopeptide, polypeptide, protein, glycoprotein, sugar and oligosaccharide;
R4 is absent or selected from a drug, —O-alkyl group, —N-alkyl group, amino acid, lipid, glycolipid, peptide, oligopeptide, polypeptide, protein, glycoprotein, sugar and oligosaccharide;
when each of R3 and R4, independently of each other, is a O- or N-alkyl group, said alkyl groups together with the O or N atoms to which they are bonded and the C2 atom may form a heterocyclic ring system, and
wherein said combination affords a water-soluble or water dispersible polymer, being substantially free of aldehyde groups.

73. The conjugate according to claim 72, comprising at least one of each of monomers (a) to (c).

74. The conjugate according to claim 72, wherein said monomer of (a) constitutes between about 10 and 98% of the weight of the conjugate.

75. The conjugate according to claim 72, wherein said oxidized form (b) constitutes between about 10 to 60% of the weight of the conjugate.

76. The conjugate according to claim 72, wherein said drug conjugate (c) comprises between about 1 to 50% of the weight of the conjugate.

77. The conjugate according to claim 72, wherein said polymer is a polysaccharide and wherein said monomer is a monosaccharide.

78. The conjugate according to claim 77, wherein said polysaccharide is selected from starch, glycogen, dextran, cellulose, pullulan, chitosan, arabinogalactan, galactan, galactomannan and guar gum.

79. The conjugate according to claim 72, wherein said oxidized form (b) is an open ring form prepared by oxidation of the monomer followed by modification thereof into a substantially aldehyde free monomer.

80. The conjugate according to claim 72, wherein said drug is a therapeutically active compound.

81. The conjugate according to claim 80, wherein said active compound is oxidation sensitive and selected from hydroxylated drugs and aminated drugs.

82. The conjugate according to claim 81, wherein said drug is selected from polyene antibiotics, low molecular weight drugs, high molecular weight drugs, amine drug derivatives, peptides, polypeptides or analogs thereof.

83. The conjugate according to claim 82, wherein said low molecular weight drug has a molecular weight of less than about 2,000 Dalton.

84. The conjugate according to claim 82, wherein said high molecular weight drug has a molecular weight of between about 2,000 and about 6000 Daltons.

85. The conjugate according to claim 81, wherein said hydroxylated drug is selected from dexamethasone, daunorubicin, cytarabine, salicylic acid, santalol, and propanolol.

86. The conjugate according to claim 82, wherein said polyene antibiotic is selected from Nystatin and Amphotericin B (AmB).

87. The conjugate according to claim 83, wherein said low molecular weight drug is selected from 5-amino salicylic acid, aminoglucoside antibiotics, polyene antibiotics, flucytosine, pyrimethamine, sulfadiazine, dapsone, trimethoprim, mitomycins, methotrexate, doxorubicin, daunorubicin, polymyxin B, propanolol, cytarabine and santalol.

88. The conjugate according to claim 82, wherein said amine drug derivative is selected from alanyl-Taxol, triglycyl-Taxol, alanyl-glycyl-dexamethasone, glycyl-dexamethasone and alanyl-dexamethasone.

89. The conjugate according to claim 82, wherein said polypeptide is selected from luteinizing hormone releasing hormone (LHRH), bradykinin, vasopressin, oxytocin, somatostatin, thyrotropin releasing factor (TRF), gonadotropin releasing hormone (GnRH), insulin and calcitonine.

90. The conjugate according to claim 72, wherein said R4 is absent or H and the N atom of said drug bonded to C1 is also bonded to C2 via a C—N single or double bond, forming a ring structure.

91. The conjugate according to claim 72, wherein said drug bonded to said polymer is selected from AmB, doxorubicin, mitomycin C, polymyxin B, paclitaxol, gentamicin, dexamethasone, 5-amino salicylic acid, and somatostatin.

92. The conjugate according to claim 91, wherein said drug is AmB and said bond is an imine or amine bond.

93. The conjugate according to claim 72,
a. wherein said R3 is OH, R4 is an O-alkyl; or
b. wherein said R3 is OH, R4 is an N-alkyl, bonded to C2 via an amine bond; or
c. wherein said R3 is absent, R4 is an N-alkyl, bonded to C2 via an imine bond; or
d. wherein said R3 is H, R4 is an O-alkyl.

94. The conjugate according to claim 72, wherein each of said R3 and R4 is, independently of each other, an O-alkyl.

95. The conjugate according to claim 72, wherein said R3 is an N-alkyl, bonded to C2 via an amine bond, and R4 is an O-alkyl.

96. The conjugate according to claim 72, wherein said R3 is H and R4 is an N-alkyl, bonded to C2 via an amine bond.

97. The conjugate according to claim 72, wherein each of said R3 and R4 independently of each other is an N-alkyl, bonded to C2 via an amine bond.

98. The conjugate according to claim 72, wherein said R3 is absent and R4 is an amino acid bonded to C2 via an imine bond.

99. The conjugate according to claim 72, wherein said R3 is H and R4 is an amino acid bonded to C2 via an amine bond.

100. The conjugate according to claim 98, wherein said amino acid is lysine.

101. The conjugate according to claim 72, wherein said R3 is absent and R4 is ═NCH₂CH₂OH.

102. The conjugate according to claim 72, wherein said R3 is H and R4 is —NZCH₂CH₂OH, wherein Z is H or an alkyl group.

103. The conjugate according to claim 72, wherein said R3 is OH and R4 is —OCH₂CH₃.

104. The conjugate according to claim 72, wherein each of said R3 and R4, independently of each other is a group which imparts said conjugate with at least one of the following characteristics: hydrophobicity, hydrophilicity, acidity, solubility, dispersability, chemical reactivity, specificity to a target tissue, modified therapeutic activity and affinity towards a certain receptor or biological active site.

105. The conjugate according to claim 104, wherein said group is selected from: (1) cholesterol and derivatives thereof; (2) glucosamine; (3) amino acids; (4) bifunctional molecules; and (5) hydrophobic groups.

106. The conjugate according to claim 72, wherein said polymer is dextran, said drug is:
a. AmB and R4 is ═NCH₂CH₂OH; or
b. AmB and R4 is —NZCH₂CH₂OH and Z is H or alkyl; or
c. AmB and R4 is —OCH₂CH₃.

107. The conjugate according to claim 72, wherein said polymer is chitosan, said drug is:
a. AmB and R4 is ═NCH₂CH₂OH; or
b. AmB and R4 is —NZCH₂CH₂OH and Z is H or alkyl; or
c. AmB and R4 is —OCH₂CH₃.

108. The conjugate according to claim 72, wherein said polymer is arabinogalactan, said drug is:
a. AmB and R4 is ═NCH₂CH₂OH; or
b. AmB and R4 is —NZCH₂CH₂OH and Z is H or alkyl; or
c. AmB and R4 is —OCH₂CH₃.

109. A method for the preparation of a conjugate according to claim 72, said method comprising:
(a) providing an unmodified water-soluble conjugate of a polymer and a drug, said polymer having at least one aldehyde group, said drug being conjugated to said polymer via a bond selected from an imine, amine, amide, ether and carboxyl bonds; and
(b) reacting said unmodified conjugate with an agent having reactivity towards said aldehyde group, and substantially no reactivity or low reactivity towards said drug or said bond;
thereby obtaining a conjugate substantially free of aldehyde groups.

110. The method according to claim 109, wherein said agent having a molecular weight lower than 500 Dalton.

111. The method according to claim 109, further comprising the step of reducing the imine bond between the drug and the polymer.

112. The method according to claim 111, wherein said polymer is a polysaccharide.

113. The method according to claim 111, wherein said conjugate substantially free of aldehyde groups has a reduced toxicity relative to the unmodified conjugate of step (a).

114. A conjugate obtained by the method according to claim 109.

115. A conjugate obtainable by the method according to claim 109.

116. A composition comprising a conjugate according to claim 72.

117. The composition according to claim 116 being a pharmaceutical composition.

118. The composition according to claim 117 being a composition selected from an antibiotic composition, an antiparasitic composition and an anticancer composition.

119. A pharmaceutical composition comprising a conjugate of a polymer and a drug according to claim 72, for the treatment of a disease or disorder treatable by said drug.

120. The composition according to claim 117, being a modified release formulation.

121. A hydrogel of a conjugate according to claim 72 and a polyamine.

122. A method for treating a disease or disorder comprising administering to a subject in need of such a treatment a conjugate according to claim 72 or a pharmaceutical composition comprising thereof.
